(19) United States
(12) Patent Application Publication
(10) Pub. No.: US 2005/0112155 A1
(43) Pub. Date: May 26, 2005

(54) Cosmetic composition comprising a passivated and optionally coated metal particles

(76) Inventors: Franck Giroud, Clichy (FR); Henri Samain, Bievres (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 10/915,461

(22) Filed: Aug. 11, 2004

Related U.S. Application Data
(60) Provisional application No. 60/511,578, filed on Oct. 16, 2003.

(30) Foreign Application Priority Data
Aug. 11, 2003 (FR) 03 50422

(51) Int. Cl. A61K 7/06; A61K 7/11

(52) U.S. Cl. 424/401; 424/70.13; 424/70.14

(57) ABSTRACT

The disclosure relates to a cosmetic composition comprising, in a physiologically acceptable medium, passivated particles predominantly comprising of at least one metal, the passivated particles treated at their outer surface with at least one carboxylate group of formula 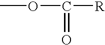 wherein, R is chosen from a linear hydrocarbon having from 6 to 40 carbons optionally comprising one or more ethylenic double bonds and, in addition, optionally being substituted by one or more substituents chosen from hydroxyl and halogen groups, and the size of the particles being less than or equal to 500 nm. The disclosure also relates to a cosmetic process for the treatment of keratinous substances comprising the application to keratinous substances, such as the hair, of the above composition.

This application claims benefit of U.S. Provisional Application No. 60/511,578, filed Oct. 16, 2003, which is herein incorporated by reference.

The disclosure relates to a cosmetic composition comprising passivated metal particles, it optionally being possible for the particles to be coated and to exhibit a core-shell structure.

The disclosure additionally relates to the coated passivated particles.

The disclosure also relates to a cosmetic process for the treatment of keratinous substances, such as the hair, for example for contributing sheen thereto, which employs the composition.

The disclosure additionally relates to a method for contributing sheen to keratinous substances, such as the hair, comprising applying the cosmetic composition disclosed herein.

The technical field of the disclosure can be defined as that of cosmetic compositions, whether hair compositions or compositions for the skin or nails.

The use of metal particles has already been disclosed in various types of cosmetic make-up compositions.

For example, the document EP-A-1 082 952 discloses make-up compositions, in particular for the nails, comprising glass particles covered with a metal layer which make it possible to obtain a make-up exhibiting a sparkling and wear-resistant metallic appearance.

The document EP-A-953 330 relates to the combination of two different compositions respectively comprising metal particles of goniochromatic pigment type and a pigment of conventional type having one of the colors of the first pigment for producing a make-up with a metallic effect which can vary according to the angle of observation and which exhibits iridescent effects.

More recently, International Patent Application WO-A-02/03913 discloses nail varnish compositions comprising particles in the form of aluminum platelets in proportions by weight of 0.4 to 0.75% and film-forming agents having high molecular weights for producing a make-up of mirror type, that is to say, in this instance, a make-up having not only the color of the aluminum but also a sheen and an ability to reflect the separate components of an object.

Metal particles have also been incorporated in hair compositions.

Thus, it is known that it is possible to contribute to the hair a better sheen than that contributed by fatty substances by incorporating metal particles, including nanoparticles, such as silver nanoparticles, in hair compositions.

Such compositions are disclosed in the document EP-A-1 064 918.

However, it has been found that the sheen contributed by such compositions fades very rapidly over time.

In another field, the document WO-A-00/78282 discloses the use of silver nanoparticles with a size of 1 to 50 nm as an antimicrobial agent in curable silicone rubber compositions.

However, this document does not disclose the use of encapsulated nanoparticles or the use of passivated nanoparticles.

There thus exists a still unfulfilled need for a cosmetic composition, such as a hair cosmetic composition, comprising metal particles which has a high sheen that is maintained over a long period of time without fading in the course of time.

There also exists a need for a cosmetic composition, such as a hair composition, which, while exhibiting a high sheen over a long period of time, also has great stability over time.

The cosmetic composition disclosed herein meets, inter alia, these needs.

One aspect disclosed herein is a cosmetic composition which does not exhibit the disadvantages, failings, limitations and inconveniences of the compositions of the prior art and which solves the problems of the compositions of the prior art.

This aspect, and still others, are achieved, in accordance with the disclosure, by a cosmetic composition comprising, in a physiologically acceptable medium, passivated particles comprising at least one metal, the particles being treated, at their outer surface, with carboxylate groups of formula (I) 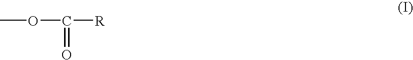
wherein R is chosen from linear hydrocarbon groups having from 6 to 40 carbons optionally comprising one or more ethylenic double bonds and, in addition, optionally being substituted by one or more substituents chosen from hydroxyl groups and halogens, including F, Br, Cl, I, wherein the size of the particles are generally less than or equal to 500 nm.

For example, in the formula (I), R is a hydrocarbon substituted by one or more fluorine atoms.

Cosmetic compositions comprising the particles incorporated in the compositions disclosed herein, have never been mentioned in the prior art. Such particles are defined by parameters chosen from structure, constituents, the agent used to treat the particles, also known as the passivating agent for the particles, and particle size.

Surprisingly, as a result of the incorporation in the compositions of these disclosed particles, which may be described as metal nanoparticles treated with or alternatively passivated by specific groups, the compositions, such as the hair compositions, make it possible to obtain a high sheen immediately after application thereof, that is to say immediately after the treatment.

However, in contrast to the compositions of the prior art which comprise different metal particles from those incorporated in the compositions disclosed herein, that is to say metal particles untreated with or non-passivated by carboxylic groups, the high sheen obtained with the compositions disclosed herein is retained for a prolonged period of time.

By way of example, this high sheen can be maintained for a period of time which can reach, for example, one month or more for hair treated with compositions disclosed herein, whereas a composition of the prior art, such as that disclosed in the document EP-A-1 064 918, including particles not in accordance with the disclosure, namely untreated or non-passivated, loses all its sheen or reflectivity after a period of 1 month.

In addition to the retention of the sheen over time, the cosmetic compositions disclosed herein exhibit a markedly better stability over time than the compositions of the prior art, for example those represented in the document EP-A-1 064 918, which comprise different metal particles from those included in the compositions disclosed herein, such as metal particles which are untreated with or non-passivated by the carboxylate groups according to the disclosure.

In addition, it may be considered that long-chain carboxylic acids, such as fatty acids, form, at the surface of the particles according to the disclosure, a passivation monolayer with a thickness generally of 1 nm to 2 nm.

This monolayer passivates the metal, namely protects the metal, such as the metal of which the particle is composed, for example from surface oxidation. The result of this is that the loss in reflectivity in the visible region and thus the loss in sheen due to this surface oxidation are avoided.

The treated or passivated metal particles which are employed in the compositions disclosed herein, in addition, optionally may be coated and can exhibit a core-shell structure in which the core comprises the treated or passivated metal particle.

Such particles, treated with or passivated by carboxylate groups and coated, have never been disclosed in the prior art and are novel.

The incorporation of these treated or passivated and coated particles in cosmetic compositions makes it possible to obtain the effects of the disclosure to an increased degree. Thus, on employing treated or passivated and coated particles, an even higher sheen is obtained for an even longer period of time and the stability of the particles is found to be further increased.

One non-limiting embodiment relates to a hair cosmetic composition, such as a hair cosmetic composition for contributing sheen to the hair.

The disclosure additionally relates to a cosmetic process for the treatment of keratinous substances, such as the hair. In one embodiment, the process comprises contributing sheen to keratinous substances including the hair, comprising applying to keratinous substances or fibers, a composition as described herein.

The disclosure also relates to the use of the particles described herein, whether passivated or passivated and coated or encapsulated, in a cosmetic composition for contributing sheen to keratinous substances or fibers, including the hair.

The disclosure will now be described in more detail in the description which follows.

The compositions according to the disclosure are defined as being cosmetic compositions.

The cosmetic compositions disclosed herein may, for example, further comprise at least one agent exhibiting a cosmetic activity or having a cosmetic effect.

The term “agent exhibiting a cosmetic activity” or “cosmetic active principle”, within the meaning of the present disclosure, is to be understood as any active compound having a cosmetic or dermatological activity or alternatively any compound capable of modifying the appearance and/or the feel and/or the physicochemical properties of keratinous substances, such as the hair.

The agents exhibiting a cosmetic activity according to the disclosure are generally chosen from:
saccharides, oligosaccharides and polysaccharides which may or may not be hydrolysed and which may or may not be modified,
amino acids, oligopeptides, peptides, proteins, which may or may not be hydrolysed and which may or may not be modified, poly(amino acid)s and enzymes,
branched and unbranched fatty acids and alcohols,
animal, vegetable and mineral waxes,
ceramides and pseudoceramides,
hydroxylated organic acids,
UV screening agents,
antioxidants and agents for combating free radicals,
chelating agents,
antidandruff agents,
seborrhoea-regulating agents,
soothing agents,
cationic surfactants,
cationic and amphoteric polymers,
organomodified and non-organomodified silicones,
mineral, vegetable and animal oils,
polyisobutenes and poly(α-olefin)s,
esters,
soluble and dispersed anionic polymers,
soluble and dispersed non-ionic polymers,
reducing agents,
coloring agents and coloring materials, such as hair dyes,
foaming agents,
film-forming agents,
particles (other than the passivated and optionally coated particles of the disclosure),
and their mixtures.

This agent exhibiting a cosmetic activity is present in a proportion ranging from 0.001 to 10% by weight of the cosmetic composition, for example from 0.01 to 5% by weight.

Generally, the compounds of saccharide, oligosaccharide or polysaccharide which may or may not be hydrolysed and which may or may not be modified and which can be used in the present disclosure may be chosen from those which are described in Encyclopedia of Chemical Technology, Kirk-Othmer, Third Edition, 1982, volume 3, pp. 896-900, and volume 15, pp. 439458; Polymers in Nature, by E. A. MacGregor and C. T. Greenwood, published by John Wiley & Sons, Chapter 6, pp. 240-328, 1980; and Industrial Gums—Polysaccharides and their Derivatives, edited by Roy L. Whistler, Second Edition, published by Academic Press Inc., the content of these three works being entirely included in the present application by way of reference.

Mention may be made, as examples of saccharides, oligosaccharides and polysaccharides which may or may not be hydrolysed and which may or may not be modified which can be used in the disclosures, of glucans, modified or unmodified starches (such as those resulting, for example, from cereals, such as wheat, maize or rice, from vegetables, such as yellow split peas, or from tubers, such as potatoes or manioc) which are different from starch betainate (starch as described above), amylose, amylopectin, glycogen, dextrans, β-glucans, celluloses and their derivatives (methylcelluloses, hydroxyalkylcelluloses, ethylhydroxyethylcelluloses or carboxymethylcelluloses), fructosans, inulin, levan, mannans, xylans, lignins, arabans, galactans, galacturonans, chitin, glucoronoxylans, arabinoxylans, xyloglucans, galactomannans, glucomannans, pectic acids and pectins, alginic acid and alginates, arabinogalactans, carrageenans, agars, glycosaminoglucans, gums arabic, gums tragacanth, ghatti gums, karaya gums, locust bean gums, guar gums and xanthan gums.

Mention may be made, as amino acids, of cysteine, lysine, alanine, N-phenylalanine, arginine, glycine, leucine, and their mixtures. Mention may be made, as oligopeptides, of peptides and proteins which may or may not be hydrolysed and which may or may not be modified which can be used according to the disclosure, of hydrolysates of wool or silk proteins, which may or may not be modified, and plant proteins, such as wheat proteins.

Mention may be made, among poly(amino acid)s which can be used, of polylysine.

Mention may be made, among enzymes which can be used, of laccases, peroxidases, lipases, proteases, glycosidases, dextranases, uricases and alkaline phosphatase.

Mention may be made, among branched and unbranched fatty acids suitable in the present disclosure, of C₈-C₃₀ carboxylic acids, such as palmitic acid, oleic acid, linoleic acid, myristic acid, stearic acid, lauric acid, and their mixtures. The fatty alcohols which can be used in the present disclosure may include C₈-C₃₀ alcohols, such as, for example, palmityl, oleyl, linoleyl, myristyl, stearyl and lauryl alcohols.

A wax within the meaning of the present disclosure may comprise a lipophilic compound, solid at ambient temperature (approximately 25° C.), with a reversible solid/liquid change of state, having a melting point of greater than approximately 40° C. and which can range up to 200° C., and exhibiting, in the solid state, an anisotropic crystalline arrangement. Generally, the size of the crystals of the wax is such that the crystals diffract and/or scatter light, conferring on the composition which comprises them a cloudy appearance which is more or less opaque. On bringing the wax to its melting point, it is possible to render it miscible with oils and to form a microscopically homogeneous mixture but, on bringing the temperature of the mixture back to ambient temperature, a recrystallization of the wax from the oils of the mixture is obtained which is detectable microscopically and macroscopically (opalescence).

Mention may be made, as waxes which can be used in the present disclosure, of waxes of animal origin, such as beeswax, spermaceti, lanolin wax and lanolin derivatives; vegetable waxes, such as carnauba wax, candelilla wax, ouricury wax, Japan wax, cocoa butter, cork fiber, and sugarcane waxes; or mineral waxes, for example paraffin wax, petrolatum wax, lignite wax or microcrystalline waxes or ozokerites.

Mention may be made, among ceramides, of ceramides of the classes I, II, III and V according to the Downing classification, such as N-oleyldehydrosphingosine.

The hydroxylated organic acids are chosen from those well known and used in the art. Mention may be made of citric acid, lactic acid, tartaric acid and malic acid.

The sunscreens active in the UV-A and/or UV-B regions which can be used according to the disclosure are those well known to a person skilled in the art. Mention may be made of dibenzoylmethane derivatives, such as 4-methyldibenzoylmethane, 4-isopropyldibenzoylmethane, 4-tert-butyldibenzoylmethane, 2,4-dimethyldibenzoylmethane and 4-tert-butyl-4′-diisopropyldibenzoylmethane; p-aminobenzoic acid and its esters, such as 2-ethylhexyl p-dimethylaminobenzoate and N-propoxylated ethyl p-aminobenzoate; salicylates, such as triethanolamine salicylate; cinnamic acid esters, such as 2-ethylhexyl 4-methoxycinnamate and methyl diisopropylcinnamate; menthyl anthranilate; benzotriazole derivatives; triazine derivatives; β,β-diphenylacrylate derivatives, such as 2-ethylhexyl 2-cyano-3,3-diphenylacrylate and ethyl 2-cyano-3,3-diphenylacrylate; 2-phenylbenzimidazole-5-sulphonic acid and its salts, benzophenone derivatives; benzylidenecamphor derivatives; silicone-comprising screening agents, and the like.

Mention may be made, as antioxidants and agents for combating free radicals which can be used in the present disclosure, of, for example, ascorbic acid; ascorbylated compounds, such as ascorbyl dipalmitate; t-butylhydroquinone; polyphenols, such as phloroglucinol; sodium sulphite; erythorbic acid and flavonoids.

The chelating agents can be chosen, for example, from EDTA (ethylenediaminetetraacetic acid) and its salts, such as disodium EDTA and dipotassium EDTA; phosphate-comprising compounds, such as sodium metaphosphate, sodium hexametaphosphate and tetrapotassium pyrophosphate; and phosphonic acids and their salts, such as the salts of ethylenediaminetetramethylenephosphonic acid.

The antidandruff agents are chosen, for example, from:
benzethonium chloride, benzalkonium chloride, chlorhexidine, chloramine-T, chloramine-B, 1,3-dibromo-5,5-dimethylhydantoin, 1,3-dichloro-5,5-di-methylhydantoin, 3-bromo-1-chloro-5,5-dimethylhydantoin and N-chlorosuccinimide;
1-hydroxy-2-pyridone derivatives, such as, for example, 1-hydroxy-4-methyl-2-pyridone, 1-hydroxy-6-methyl-2-pyridone and 1-hydroxy-4,6-dimethyl-2-pyridone;
trihalocarbamides;
triclosan;
azole-comprising compounds, such as climbazole, ketoconazole, ciotrimazole, econazole, isoconazole and miconazole b;
antifungal polymers, such as amphotericin B or nystatin;
selenium sulphides;
sulphur in its various forms, cadmium sulphide, allantoin, coal or wood tars and their derivatives, such as oil of cade, undecylenic acid, fumaric acid, and allylamines, such as terbinafine.

They may also be used in the form of their addition salts with physiologically acceptable acids, for example, in the form of salts of sulphuric, nitric, thiocyanic, hydrochloric, hydrobromic, hydriodic, phosphoric, acetic, benzoic, glycolic, aceturic, succinic, nicotinic, tartaric, maleic, palmitic, methanesulphonic, propanoic, 2-oxopropanoic, propanedioic, 2-hydroxy-1,4-butanedioic, 3-phenyl-2-propenoic, α-hydroxybenzeneacetic, ethanesulphonic, 2-hydroxyethanesulphonic, 4-methylbenzenesulphonic, 4-amino-2-hydroxybenzoic, 2-phenoxybenzoic, 2-acetyloxybenzoic, picric, lactic, citric, malic and oxalic acids and of amino acids.

The antidandruff agents mentioned above can also, if appropriate, be used in the form of their addition salts with physiologically acceptable organic or inorganic bases. Non-limiting examples of organic bases are those chosen from alkanolamines with low molecular weights, such as ethanolamine, diethanolamine, N-ethylethanolamine, triethanolamine, diethylaminoethanol and 2-amino-2-methylpropanedione; nonvolatile bases, such as ethylenediamine, hexamethylenediamine, cyclohexylamine, benzylamine and N-methylpiperazine; quaternary ammonium hydroxides, for example trimethylbenzylammonium hydroxide; and guanidine and its derivatives, for example its alkylated derivatives. Examples of inorganic bases include the salts of alkali metals, such as sodium and potassium; ammonium salts; the salts of alkaline earth metals, such as magnesium or calcium; and the salts of cationic di-, tri- or tetravalent metals, such as zinc, aluminum and zirconium. Alkanolamines, ethylenediamine and inorganic bases, such as the salts of alkali metals may be used.

The seborrhoea-regulating agents are, for example, succinylchitosan and poly-β-alanine.

The soothing agents are, for example, azulene and glycyrrhetinic acid.

The cationic surfactants are those well known, such as salts of primary, secondary or tertiary fatty amines which are optionally polyoxyalkylenated; quaternary ammonium salts, such as tetraalkylammonium, alkylamidoalkyltrialkylammonium, trialkylbenzylammonium, trialkylhydroxyalkylammonium and alkylpyridinium chlorides and bromides; and imidazoline derivatives.

The term “cationic polymer” is understood to mean any polymer comprising cationic groups and/or groups which can be ionized to cationic groups.

The cationic polymers which can be used in accordance with the present disclosure can be chosen from all those already known for improving the cosmetic properties of hair treated with detergent compositions, for example those disclosed in Patent Application EP-A-0 337 354 and in French Patent Applications FR-A-2 270 846, 2 383 660, 2 598 611, 2 470 596 and 2 519 863.

The cationic polymers may be chosen from, for example, those which comprise units comprising primary, secondary, tertiary and/or quaternary amine groups which can either form part of the main polymer chain or be carried by a side substituent directly connected to the main chain.

The cationic polymers used generally have a number-average molecular mass ranging from 500 to 5×10⁶, for example from 10³ to 3×10⁶.

Mention may also be made, among cationic polymers, of the polymers of the polyamine, polyaminoamide and poly(quaternary ammonium) type. These are known products.

The polymers of the polyamine, polyaminoamide or poly(quaternary ammonium) type which can be used in the composition disclosed herein are those disclosed in French Patents Nos. 2 505 348 and 2 542 997. Mention may be made, among these polymers, for example, of:
(1) homopolymers and copolymers derived from esters and amides of acrylic and methacrylic acid;
(2) cellulose ether derivatives comprising quaternary ammonium groups disclosed in French Patent 1 492 597;
(3) cationic cellulose derivatives, such as the copolymers of cellulose or the cellulose derivatives grafted with a water-soluble quaternary ammonium monomer and disclosed in U.S. Pat. No. 4,131,576, such as hydroxyalkylcelluloses, for example hydroxymethyl-, hydroxyethyl- and hydroxypropyl-celluloses, grafted, for example, with a methacryloylethyltrimethylammonium, methacrylamido-propyltrimethylammonium or dimethyldiallylammonium salt;
(4) the cationic polysaccharides disclosed, for example, in U.S. Pat. Nos. 3,589,578 and 4,031,307, such as guar gums comprising trialkylammonium cationic groups;
(5) polymers composed of piperazinyl units and of divalent, straight- or branched-chain alkylene or hydroxyalkylene groups, optionally interrupted by oxygen, sulphur or nitrogen atoms or by aromatic or heterocyclic rings, as well as the oxidation and/or quaternization products of these polymers. Such polymers are disclosed, for example, in French Patents 2 162 025 and 2 280 361;
(6) water-soluble polyaminoamides, such as those disclosed in French Patents 2 252 840 and 2 368 508;
(7) polyaminoamide derivatives, for example the adipic acid/dialkylaminohydroxyalkyldialkylenetriamine polymers in which the alkyl group comprises from 1 to 4 carbon atoms, for example a methyl, ethyl or propyl group and the alkylene group comprises from 1 to 4 carbon atoms, for example the ethylene group. Such polymers are disclosed in French Patent 1 583 363.
(8) polymers obtained by reaction of a polyalkylenepolyamine comprising two primary amine groups and at least one secondary amine group with a dicarboxylic acid chosen from diglycolic acid and saturated aliphatic dicarboxylic acids having from 3 to 8 carbon atoms. The molar ratio of polyalkylenepolyamine to dicarboxylic acid ranges from 0.8:1 to 1.4:1; the polyaminoamide resulting therefrom being reacted with epichlorohydrin in a molar ratio of epichlorohydrin with respect to the secondary amine group of the polyaminoamide ranging from 0.5:1 to 1.8:1. Such polymers are disclosed, for example, in U.S. Nos. 3 227 615 and 2 961 347.
(9) cyclopolymers of alkyldiallylamine or of dialkyldiallylammonium, such as the homopolymer of dimethyldiallylammonium chloride and the copolymers of diallyldimethylammonium chloride and of acrylamide;
(10) the quaternary diammonium polymers exhibiting a number-average molecular mass generally ranging from 1000 to 100 000, such as those disclosed, for example, in French Patents 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020;
(11) polymers of poly(quaternary ammonium)s, such as those disclosed in Patent Application EP-A-122 324;
(12) quaternary polymers of vinylpyrrolidone and of vinylimidazole, such as, for example, the products sold under the names Luviquat® FC 905, FC 550 and FC 370 by BASF;
(13) polyamines, such as Polyquart® H sold by Henkel, referenced under the name of “Polyethylene Glycol (15) Tallow Polyamine” in the CTFA dictionary;
(14) crosslinked polymers of methacryloyloxy(C₁-C₄)alkyltri(C₁-C₄)alkylammonium salts, such as those sold under the names of Salcare® SC 92, Salcare® SC 95 and Salcare® SC 96 by Allied Colloids; and
   their mixtures.

Other cationic polymers which can be used in the context of the invention are cationic proteins or cationic protein hydrolysates, polyalkyleneimines, for example polyethyleneimines, polymers comprising vinylpyridine or vinylpyridinium units, condensates of polyamines and of epichlorohydrin, quaternary polyureylenes and chitin derivatives.

The amphoteric polymers which can be used in accordance with the disclosure can be chosen from polymers comprising B and C units distributed randomly in the polymer chain, where B comprises a unit derived from a monomer comprising at least one nitrogen atom and C comprises a unit derived from an acidic monomer comprising one or more carboxyl or sulpho groups. Alternatively, B and C can be chosen from groups derived from zwitterionic carboxybetaine and sulphobetaine monomers. B and C can also be chosen from a cationic polymer chain comprising primary, secondary, tertiary or quaternary amine groups, in which at least one of the amine groups carries a carboxyl or sulpho group connected via a hydrocarbon. In one embodiment, B and C may form part of a chain of a polymer comprising a dicarboxyethylene unit, one of the carboxyl groups of which has been reacted with a polyamine comprising one or more primary or secondary amine groups.

The amphoteric polymers corresponding to the definition given above may be chosen from, for example, the following polymers:
(1) polymers resulting from the copolymerization of a monomer derived from a vinyl compound carrying a carboxyl group, such as acrylic acid, methacrylic acid, maleic acid and α-chloroacrylic acid, and of a basic monomer derived from a substituted vinyl compound comprising at least one basic atom, such asdialkylaminoalkyl methacrylates and acrylates or dialkylaminoalkylmethacrylamides and -acrylamides. Such compounds are disclosed in U.S. Pat. No. 3,836,537. Mention may also be made of the sodium acrylate/acrylamidopropyltrimethylammonium chloride copolymer sold under the name Polyquart® KE 3033 by Henkel. The vinyl compound can also be a dialkyldiallylammonium salt, such as diethyldiallylammonium chloride. Copolymers of acrylic acid and of the latter monomer are provided under the names Merquat® 280, Merquat® 295 and Merquat® Plus 3330 by Calgon.
2) polymers comprising units deriving:
a) from at least one monomer chosen from acrylamides and methacrylamides substituted on the nitrogen by an alkyl group,
b) from at least one acidic comonomer comprising one or more reactive carboxyl groups, and
c) from at least one basic comonomer, such as esters comprising primary, secondary, tertiary and quaternary amine substituents of acrylic and methacrylic acids and the quaternization product of dimethylaminoethyl methacrylate with dimethyl or diethyl sulphate.

The N-substituted acrylamides or methacrylamides according to the disclosure may be, for example the groups in which the alkyl groups comprise from 2 to 12 carbon atoms, such as N-ethylacrylamide, N-tert-butylacrylamide, N-tert-octylacrylamide, N-octylacrylamide, N-decylacrylamide or N-dodecylacrylamide, and the corresponding methacrylamides.

The acidic comonomers may be chosen from, for example, acrylic, methacrylic, crotonic, itaconic, maleic, fumaric acids and alkyl monoesters, having 1 to 4 carbon atoms, of maleic or fumaric acids or anhydrides. The basic comonomers may be, for example, aminoethyl, butylaminoethyl, N,N-dimethylaminoethyl or N-tert-butylaminoethyl methacrylates. Use may be made, for example, of the copolymers for which the CTFA name (4th Ed., 1991) is octylacrylamide/acrylates/butylaminoethyl methacrylate copolymer, such as the products sold under the name Amphomer® or Lovocryl® 47 by National Starch.
(3) partially or completely alkylated and crosslinked polyaminoamides deriving from polyaminoamides of general formula:
   CO—R₁₀—CO-Z (III)
   wherein R₁₀ comprises a divalent group derived from a group chosen from saturated dicarboxylic acid, from an aliphatic mono- or dicarboxylic acid comprising an ethylenic double bond, from an ester of a lower alkanol having 1 to 6 carbon atoms of these acids, and from a group deriving from the addition of any one of the acids with a bisprimary or bis(secondary derived)amine, and Z comprises a group of a bisprimary, mono- or bissecondary polyalkylenepolyamine and may, for example, be chosen from:
a) in proportions ranging from 60 to 100 mol %, the group
   —NH(CH₂)ₓ—NHₚ (IV)
   where x=2 and p=2 or 3, or else x=3 and p=2, this group deriving from diethylenetriamine, triethylenetetraamine or dipropylenetriamine;
b) in proportions ranging from 0 to 40 mol %, the above group (IV), in which x=2 and p=1 and which derives from ethylenediamine, or the group deriving from piperazine: 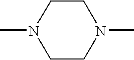
c) in proportions ranging from 0 to 20 mol %, the group —NH—(CH₂)₆—NH— deriving from hexamethylenediamine, these polyaminoamides being crosslinked by addition of a bifunctional crosslinking agent chosen from epihalohydrins, diepoxides, dianhydrides and bisunsaturated derivatives, by means of 0.025 to 0.35 mol of crosslinking agent per amine group of the polyaminoamide, and alkylated by reaction with an agent chosen from acrylic acid, chloracetic acid, an alkanesultone, and their salts.

The saturated carboxylic acids may be chosen from, for example, acids having 6 to 10 carbon atoms, such as adipic, 2,2,4-trimethyladipic and 2,4,4-trimethyladipic, and terephthalic acids, and the acids comprising an ethylenic double bond, such as, for example, acrylic, methacrylic and itaconic acids. The alkanesultones used in the alkylation may be, for example, propane- or butanesultone and the salts of the alkylating agents may be, for example, the sodium or potassium salts.
(4) polymers comprising zwitterionic units of formula: 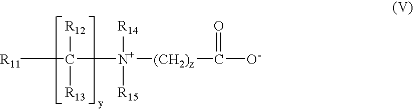
   wherein
   R₁₁ is chosen from a polymerizable unsaturated group, such as a group chosen from acrylates, methacrylates, acrylamides and methacrylamides,
   y and z each are an integer from 1 to 3,
   R₁₂ and R₁₃ are chosen from hydrogen atoms, methyl groups, ethyl groups, and propyl groups, and
   R₁₄ and R₁₅ are chosen from hydrogen atoms and alkyl groups such that the sum of the carbon atoms in R₁₄ and R₁₅ does not exceed 10.

The polymers comprising such units can also comprise units derived from non-zwitterionic monomers, such as those chosen from dimethyl- and diethylaminoethyl, acrylate and methacrylate, alkyl acrylates and methacrylates, acrylamides and methacrylamides, and vinyl acetate.

Mention may be made, by way of example, of the copolymer of methyl methacrylate and of dimethylcarboxymethylammonioethyl methacrylate, such as the product sold under the name Diaformer® Z301 by Sandoz.
(5) polymers derived from chitosan comprising monomer units corresponding to the following formulae: 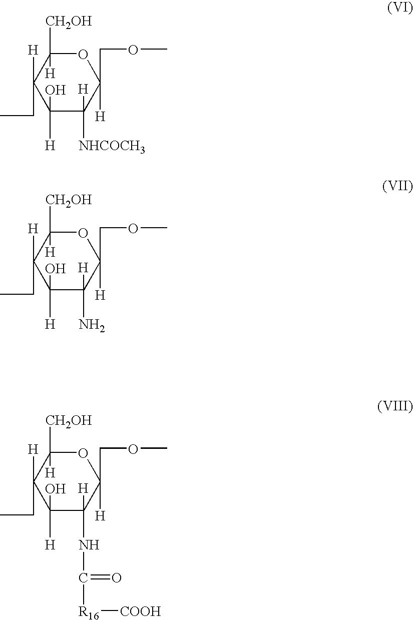
   the unit (VI) being present in proportions ranging from 0 to 30%, in weight percent, the unit (VII) being present in proportions ranging from 5 to 50%, in weight percent, and the unit (VIII) being present in proportions ranging from 30 to 90%, in weight percent, it being understood that, in this unit (VIII), R₁₆ comprises a group of formula: 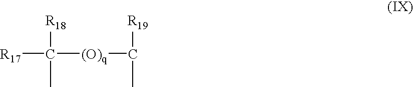
   wherein
   if q=0, R₁₇, R₁₈ and R₁₉, which may be identical or different, are independently chosen from hydrogen atoms, methyl residues, hydroxyl residues, acetoxy residues, amino residues, monoalkylamino residues, and dialkylamino residues, optionally interrupted by at least one nitrogen atom and/or optionally substituted by at least one group chosen from aminos, hydroxyls, carboxyls, alkylthios and sulpho groups, and alkylthio residues wherein the alkyl group carries an amino residue, at least one of the R₁₇, R₁₈ and R₁₉ groups being, in this case, a hydrogen atom;
      alternatively
   if q=1, R₁₇, R₁₈ and R₁₉ are chosen from hydrogen atoms, and the salts formed by these compounds with bases or acids.
(6) polymers derived from the N-carboxyalkylation of chitosan, such as the N-(carboxymethyl)chitosan and the N-(carboxybutyl)chitosan sold under the name Evalsan® by Jan Dekker.
(7) polymers corresponding to the general formula (X) disclosed, for example, in French Patent 1 400 366: 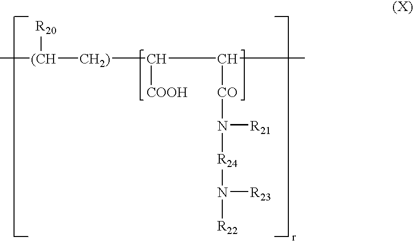
   wherein
   R₂₀ is chosen from a hydrogen atom, a CH₃O group, a CH₃CH₂O group, and a phenyl group,
   R₂₁ is chosen from a hydrogen and a lower alkyl group, such as methyl or ethyl,
   R₂₂ is chosen from a hydrogen and a lower alkyl group, such as methyl or ethyl, and
   R₂₃ is chosen from a lower alkyl group, such as a methyl or ethyl, and a group corresponding to the formula: —R₂₄—N(R₂₂)₂, wherein R₂₄ is chosen from a —CH₂—CH₂—, —CH₂—CH₂—CH₂— and a —CH₂—CH(CH₃)— group, wherein R₂₂ has the meanings mentioned above, as well as the higher homologues of these groups comprising up to 6 carbon atoms.
(8) amphoteric polymers of the -D-X₁-D-X₁— type chosen from:
a) polymers obtained by the reaction of chloroacetic acid or sodium chloroacetate with compounds comprising at least one unit of formula (XI):
   -D-X₁-D-X₁-D- (XI)
   wherein
   D comprises the group 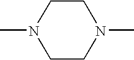
   and X₁ is represented by the symbol E or E′, which may be identical or different, and are chosen from a bivalent group which comprises a straight- or branched-chain alkylene group comprising up to 7 carbon atoms in the main chain which may be unsubstituted or substituted by one or more hydroxyl groups and which can further comprise oxygen, nitrogen or sulphur atoms or 1 to 3 aromatic and/or heterocyclic rings; the oxygen, nitrogen and sulphur atoms being present in the form chosen from ether, thioether, sulphoxide, sulphone, sulphonium, alkylamine and alkenylamine groups and hydroxyl, benzylamine, amine oxide, quaternary ammonium, amide, imide, alcohol, ester and urethane groups.
b) polymers of formula:
   -D-X₁-D-X₁— (XII)
   wherein
   D comprises the group 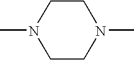
   and X₁ is represented by the symbol E or E′ and having the meaning indicated above. For example, E or E′, which may be identical or different, and are chosen from a bivalent group which is a straight- or branched-chain alkylene group having up to 7 carbon atoms in the main chain which may be unsubstituted or substituted by one or more hydroxyl groups and which comprises one or more nitrogen atoms, the nitrogen atom optionally being substituted by an alkyl chain optionally interrupted by an oxygen atom and comprising one or more functional groups chosen from carboxyls, hydroxyls and betainized by reaction with at least one compound chosen from chloroacetic acid and sodium chloroacetate.
(9) (C₁-C₅)alkyl vinyl ether/maleic anhydride copolymers partially modified by semiamidation with an N,N-dialkylaminoalkylamine, such as N,N-dimethylamino-propylamine, or by semiesterification with an N,N-dialkanolamine. These copolymers can also comprise other vinyl comonomers, such as vinylcaprolactam.

The silicones which can be used in accordance with the disclosure can be soluble or insoluble in water and they may be, for example, polyorganosiloxanes which are insoluble in water. The silicones can be provided in the form of oils, waxes, resins or gums.

Organopolysiloxanes are defined in more detail in the work by Walter Noll, Chemistry and Technology of Silicones (1968), Academic Press. Such organopolysiloxanes can be volatile or non-volatile.

When they are volatile, the silicones may be chosen from, for example, those having a boiling point ranging from 60° C. to 260° C., such as from:
(i) cyclic silicones comprising from 3 to 7 silicon atoms, for example 4 to 5 silicon atoms. Such cyclic silicones include, for example, octamethylcyclotetrasiloxane, sold, for example under the name of “Volatile Silicone 7207” by Union Carbide or “Silbione 70045 V 2” by Rhodia; or decamethylcyclopentasiloxane, sold under the name “Volatile Silicone 7158” by Union Carbide or “Silbione 70045 V 5” by Rhodia.

Mention may also be made of cyclocopolymers of the dimethylsiloxane/methylalkylsiloxane type, such as “Silicone Volatile FZ 3109”, sold by Union Carbide, with the chemical structure: with D: 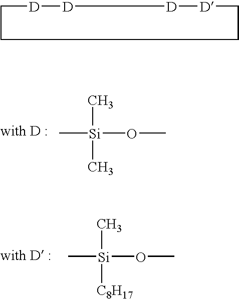
(ii) linear volatile silicones having 2 to 9 silicon atoms and having a viscosity of less than or equal to 5×10⁻⁶ m²/s at 25° C. Non-limiting examples of such silicones, include, for example, decamethyltetrasiloxane, sold under the name “SH 200” by Toray Silicone. Silicones coming within this class are also described in the article published in Cosmetics and Toiletries, Vol. 91, Jan. 76, p. 27-32, Todd & Byers, “Volatile Silicone Fluids for Cosmetics”.

Mention may be made, among nonvolatile silicones, of polyalkylsiloxanes, polyarylsiloxanes, polyalkylarylsiloxanes, silicone gums and resins, and polyorganosiloxanes modified by organofunctional groups.

The organomodified silicones which can be used in accordance with the invention are silicones as defined above and comprising, in their structure, one or more organofunctional groups attached via a hydrocarbon.

Mention may be made, among the organomodified silicones, of polyorganosiloxanes comprising:
polyethyleneoxy and/or polypropyleneoxy groups optionally comprising C₆-C₂₄ alkyl groups, such as the products known as dimethicone copolyol sold by Dow Corning under the name DC 1248 or the Silwet® L 722, L 7500, L 77 and L 711 oils from Union Carbide and the (C₁₂)alkyl methicone copolyol sold by Dow Corning under the name Q2 5200;
substituted or unsubstituted amino groups, such as the products sold under the names GP 4 Silicone Fluid and GP 7100 by Genesee or the products sold under the names Q2 8220 and Dow Corning 929 or 939 by Dow Corning. The substituted amino groups may be, for example, C₁-C₄ aminoalkyl groups;
thiol groups, such as the products sold under the names “GP 72 A” and “GP 71” from Genesee;
alkoxylated groups, such as the product sold under the names “Silicone Copolymer F-755” by SWS Silicones and Abil Wax® 2428, 2434 and 2440 by Goldschmidt;
hydroxylated groups, such as the polyorganosiloxanes comprising a hydroxyalkyl functional group disclosed in French Patent Application FR-A-85 16334;
acyloxyalkyl groups, such as, for example, the polyorganosiloxanes disclosed in U.S. Pat. No. 4,957,732 A;
anionic groups of the carboxylic acid type, such as, for example, the products disclosed in Patent EP 186 507 from Chisso Corporation; or of the alkylcarboxylic type, such as those present in the product X-22-3701 E from Shin-Etsu; 2-hydroxyalkylsulphonate or 2-hydroxyalkyl thiosulphate, such as the products sold by Goldschmidt under the names “Abil® S201” and “Abil® S255”;
hydroxyacylamino groups, such as the polyorganosiloxanes disclosed in application EP 342 834. Mention may be made, for example, of the product Q2-8413 from Dow Corning.

Mention may be made, as oils of vegetable origin, of sweet almond oil, avocado oil, castor oil, olive oil, jojoba oil, sunflower oil, wheat germ oil, sesame oil, groundnut oil, grape seed oil, soybean oil, rapeseed oil, safflower oil, coconut oil, maize oil, hazelnut oil, karite butter, palm oil, apricot kernel oil, and calophyllum oil; as oils of animal origin, of perhydrosqualene; as oils of mineral origin, of liquid paraffin and liquid petrolatum.

The polyisobutenes and poly(α-olefin)s are chosen from those well known in the art.

Mention may be made, as examples of esters, of esters of fatty acids, such as isopropyl myristate, isopropyl palmitate, 2-ethylhexyl palmitate, purcellin oil (stearyl octanoate), isononyl isononanoate, isostearyl isononanoate, isopropyl lanolate, and their mixtures.

The anionic polymers generally used in the present disclosure are polymers comprising groups derived from functional units chosen from carboxylic, sulphonic and phosphoric acids and exhibiting a weight-average molecular mass ranging from 500 to 5 000 000.

The carboxyl groups are comprised of unsaturated carboxylic monoacid or diacid monomers, such as those corresponding to the formula: 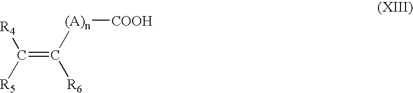
wherein
n is an integer ranging from 0 to 10,
A comprises a methylene group, optionally connected to the carbon atom of the unsaturated group or to the neighboring methylene group when n is greater than 1 via a heteroatom, such as oxygen or sulphur,
R₄ is chosen from a hydrogen atom, phenyl groups, and benzyl groups,
R₅ is chosen from a hydrogen atom, lower alkyl groups and carboxyl groups, and
R₆ is chosen from a hydrogen atom, lower alkyl groups, —CH₂—COOH groups, phenyl groups, and benzyl groups.

In the above formula (XIII), the lower alkyl groups comprise, for example, from 1 to 4 carbon atoms, such as methyl and ethyl groups.

The anionic polymers comprising carboxyl groups according to the disclosure may be, for example:
A) Homo- or copolymers of acrylic or methacrylic acid or their salts such as the products sold under the names Versicol® E or K by Allied Colloid or Ultrahold® by BASF, the copolymers of acrylic acid and acrylamide sold in the form of their sodium salt under the names Reten® 421, 423 or 425 by Hercules or the sodium salts of polyhydroxycarboxylic acids.
B) Copolymers of acrylic acid or methacrylic acid with a monoethylenic monomer, such as ethylene, styrene, vinyl esters or esters of acrylic or methacrylic acid, optionally grafted onto a polyalkylene glycol, such as polyethylene glycol, and optionally crosslinked. Such polymers are disclosed, for example, in French Patent 1 222 944 and German Application No. 2 330 956, the copolymers of this type comprising, in their chain, an optionally N-alkylated and/or -hydroxyalkylated acrylamide unit, such as disclosed in Luxembourgian Patent Applications 75370 and 75371 or provided under the name Quadramer® by American Cyanamid. Mention may also be made of copolymers of acrylic acid and of C₁-C₄ alkyl methacrylate and the copolymer of methacrylic acid and of ethyl acrylate sold under the name Luvimer® MAEX by BASF.
C) Copolymers derived from crotonic acid, such as those comprising, in their chain, vinyl acetate or propionate units and optionally other monomers, such as allyl or methallyl ester, vinyl ether or vinyl esters of a linear and branched saturated carboxylic acids comprising a long hydrocarbon chain, such as those comprising at least 5 carbon atoms, these polymers optionally being grafted and crosslinked, or alternatively a vinyl, allyl or methallyl ester of an α- or β-cyclic carboxylic acid. Such polymers are disclosed, inter alia, in French patents numbers 1 222 944, 1 580 545, 2 265 782, 2 265 781, 1 564 110 and 2 439 798. Commercial products coming within this class are the Resins 28-29-30, 26-13-14 and 28-13-10 sold by National Starch.
D) Polymers derived from maleic, fumaric or itaconic acids or anhydrides with vinyl esters, vinyl ethers, vinyl halides, phenylvinyl derivatives, or acrylic acid and its esters; these polymers can be esterified. Such polymers are disclosed, for example, in U.S. Pat. Nos. 2,047,398, 2,723,248 and 2,102,113 and Patent GB 839 805 and those sold under the names Gantrez® AN or ES by ISP.

Polymers also coming within this class are copolymers of maleic, citraconic or itaconic anhydrides and of an allyl or methallyl ester, optionally comprising an acrylamide or methacrylamide group, an α-olefin, acrylic or methacrylic esters, acrylic or methacrylic acids, or vinylpyrrolidone in their chain; the anhydride functional groups are monoesterified or monoamidated. These polymers are, for example, disclosed in French Patents 2 350 384 and 2 357 241 of the Applicant.
E) Polyacrylamides comprising carboxylate groups.

The polymers comprising sulpho groups are polymers comprising functional units chosen from vinylsulphonic, styrenesulphonic, naphthalenesulphonic and acrylamidoalkylsulphonic units.

These polymers can, for example, be chosen from:
salts of polyvinylsulphonic acid having a molecular mass ranging from 1000 to 100 000, as well as copolymers with an unsaturated comonomer, such as acrylic or methacrylic acids and their esters, as well as acrylamide or its derivatives, vinyl ethers and vinylpyrrolidone;
salts of polystyrenesulphonic acid, the sodium salts having a molecular mass of 500 000 and of 100 000 sold respectively under the names Flexan® 500 and Flexan® 130 by National Starch. These compounds are disclosed in French Patent No. 2 198 719;
salts of polyacrylamidosulphonic acids, such as those mentioned in U.S. Pat. No. 4,128,631, for example the polyacrylamidoethylpropanesulphonic acid sold under the name Cosmedia Polymer® HSP 1180 by Henkel.

According to the disclosure, the anionic polymers may be chosen from, for example, acrylic acid copolymers, such as the acrylic acid/ethyl acrylate/N-tert-butylacrylamide terpolymer sold under the name Ultrahold Strong® by BASF, copolymers derived from crotonic acid, such as the vinyl acetate/vinyl tert-butylbenzoate/crotonic acid terpolymers and the crotonic acid/vinyl acetate/vinyl neododecanoate terpolymers sold under the name Resin 28-29-30 by National Starch, polymers derived from maleic, fumaric or itaconic acids or anhydrides with vinyl esters, vinyl ethers, vinyl halides, phenylvinyl derivatives, or acrylic acid and its esters, such as the monoesterified methyl vinyl ether/maleic anhydride copolymer sold under the name Gantrez® ES 425 by ISP, copolymers of methacrylic acid and of methyl methacrylate sold under the name Eudragit® L by Rohm Pharma, the copolymer of methacrylic acid and of ethyl acrylate-sold under the name Luvimer® MAEX by BASF, the vinyl acetate/crotonic acid copolymer sold under the name Luviset® CA 66 by BASF and the vinyl acetate/crotonic acid/polyethylene glycol terpolymer sold under the name Aristoflex® A by BASF.

According to the disclosure, the anionic polymers can also be used in the latex or pseudolatex form, that is to say in the form of an aqueous dispersion of insoluble polymer particles.

Mention may be made, as non-ionic polymers which can be used according to the present disclosure, of:
vinylpyrrolidone homopolymers;
copolymers of vinylpyrrolidone and of vinyl acetate;
polyalkyloxazolines, such as the polyethyloxazolines provided by Dow Chemical under the names PEOX® 50 000, PEOX® 200 000 and PEOX® 500 000;
vinyl acetate homopolymers, such as the product provided under the name Appretan® EM by Hoechst or the product provided under the name Rhodopas® A 012 by Rhône-Poulenc;
copolymers of vinyl acetate and of acrylic ester, such as the product provided under the name Rhodopas® AD 310 from Rhone-Poulenc;
copolymers of vinyl acetate and of ethylene, such as the product provided under the name Appretan® TV by Hoechst;
copolymers of vinyl acetate and of maleic ester, for example of dibutyl maleate, such as the product provided under the name Appretan® MB Extra by Hoechst;
copolymers of polyethylene and of maleic anhydride;
alkyl acrylate homopolymers and alkyl methacrylate homopolymers, such as the product provided under the name Micropearl® RQ 750 by Matsumoto or the product provided under the name Luhydran® A 848 S by BASF;
acrylic ester copolymers, such as, for example, copolymers of alkyl acrylates and of alkyl methacrylates, such as the products provided by Rohm & Haas under the names Primal® AC-261 K and Eudragit® NE 30 D, by BASF under the names Acronal® 601, Luhydran® LR 8833 or 8845, or by Hoechst under the names Appretan® N 9213 or N9212;
copolymers of acrylonitrile and of a non-ionic monomer chosen, for example, from butadiene and alkyl (meth)acrylates; mention may be made of the products provided under the names Nipol® LX 531 8 by Nippon Zeon or those provided under the name CJ 0601 8 by Rohm & Haas;
polyurethanes, such as the products provided under the names Acrysol® RM 1020 or Acrysol® RM 2020 by Rohm & Haas or the products Uraflex® XP 401 UZ or Uraflex® XP 402 UZ provided by DSM Resins;
copolymers of alkyl acetate and of urethane, such as the product 8538-33 provided by National Starch;
polyamides, such as the product Estapor® LO 11 provided by Rhone-Poulenc;
chemically modified or unmodified non-ionic guar gums.

The unmodified non-ionic guar gums may be, for example, the products sold under the name Vidogum® GH 175 by Unipectine and under the name Jaguar® C by Meyhall.

The modified non-ionic guar gums which can be used according to the disclosure may be modified, for example, with C₁-C₆ hydroxyalkyl groups. Mention may be made, by way of examples, of the hydroxymethyl, hydroxyethyl, hydroxypropyl and hydroxybutyl groups.

These guar gums are well known in the state of the art and can, for example, be prepared by reacting corresponding alkene oxides, such as, for example, propylene oxides, with guar gum, so as to obtain a guar gum modified with hydroxypropyl groups.

Such non-ionic guar gums, optionally modified with hydroxyalkyl groups, are, for example, sold under the trade names Jaguar® HP8, Jaguar® HP60, Jaguar® HP120, Jaguar® DC 293 and Jaguar® HP 105 by Meyhall or under the name Galactasol® 4H4FD2 by Aqualon.

The alkyl groups of the non-ionic polymers may comprise, for example, from 1 to 6 carbon atoms.

The reducing agents can be chosen from thioacids and their salts (thioglycolic acid or thiosulphate, cysteine or cysteamine), alkali metal or alkaline earth metal sulphites, reducing sugars, such as glucose, vitamin C and its derivatives, sulphovinic (ethyl sulphuric) acid derivatives, or phosphines.

The coloring agents can be chosen from linear or aromatic (heterocyclic or nonheterocylic) conjugated structures. Mention may be made, among these, of nitrobenzene dyes, aromatic dyes, aminobenzene dyes, azo dyes, anthraquinone dyes, aromatic diamines, aminophenols, phenols and naphthols, porphyrins, tetra-phenylporphyrins, metalloporphyrins, phthalocyanines, carotenoids, flavonoids and fluorescent molecules (fluorescein, rhodamine, coumarin, and the like).

The film-forming agents can be chosen from film-forming polymers, for example those disclosed in FR-2 739 022, FR-2 757 048 or FR-2 767 699 belonging to L'Oréal.

The foaming agents can be generally chosen from surfactants with a foaming nature; cationic polymers and anionic polymers with foaming properties. Optionally, the foaming agent can be a specific agent, such as that disclosed in FR-2 751 221 belonging to L'Oréal.

The particles, as cosmetic active principles, may comprise other than the particles with a core-shell structure of the disclosure and can be chosen from organic, inorganic and composite particles.

The cosmetic compositions according to the disclosure are essentially characterized by the particles which they comprise.

These particles are, according to one embodiment of the disclosure, particles which can be defined as being metal nanoparticles passivated by carboxylate groups and optionally encapsulated by, for example, an organic material.

Within the meaning of the present disclosure, the term “nanoparticles” is to be understood as indicating particles with a size of less than or equal to 500 nm, for example, having a size ranging from 1 nm to 500 nm, such as from 1 nm to 100 nm, for further example 1 nm and 50 nm.

The term “particle size” is understood to mean the maximum dimension which it is possible to measure between two points of the particle. Such sizes can be measured directly by microscopic techniques, such as scanning electron microscopy or atomic force microscopy, or by indirect techniques, such as dynamic light scattering.

The particles incorporated in the compositions disclosed herein can have various shapes. They can, for example, assume the shape chosen from spheres, flakes, fibers, tubes, and polyhedra. They can also have an entirely random shape, e.g., a shape not within any of the previously mentioned shapes. In one embodiment, they can have a spherical shape.

The passivated particles incorporated in the compositions disclosed herein are predominantly comprised of a metal.

The term “metal” is understood to mean a simple body composed solely of atoms of a metal element capable of generating cations.

In the case where the passivated particles are coated or encapsulated and have a core-shell structure, the passivated particle constitutes the core of the passivated and coated particle with a core-shell structure and this core is thus predominantly composed of at least one metal.

The term “predominantly” is understood to mean that the particle is comprised of 50% or more by weight of at least one metal.

The particle may be, for example, comprised of at least 80% by weight, such as at least 90% by weight and for further example 100% by weight of at least one metal.

The term “metal” generally means aluminum and all the elements with an atomic number ranging from 21 to 82 and composing Groups 3 to 13 of the Periodic Table of the Elements according to the new IUPAC notation: reference may be made, on this subject, to the CRC Handbook of Chemistry and Physics, 80th Print Edition.

The term “metal” also includes all the alloys of these elements, and the mixtures of these metals and alloys.

The particle can thus also comprise, in the abovementioned percentages, of a mixture of two or more of these metals and/or alloys of the latter.

The particle can also be a composite particle composed of several regions, adjacent regions being composed of different metals, alloys or mixtures.

Composite particles may be, for example, multilayer particles comprising an inner core forming a substrate comprised of a metal, alloy or mixture of metals or alloys, at least partially covered by a first layer made of a metal, metal alloy or mixture of metals or alloys which is different from that constituting the inner core and optionally by one or more other layers, each of these layers at least partially covering the preceding layer and each layer being composed of a metal, alloy or mixture of metals or alloys which is different from the following layer (if the latter exists) and from the preceding layer.

Apart from the metal or metals, the particle can additionally comprise stabilizers of any kind and unavoidable impurities.

The particle can also comprise, for example, metal compounds other than metals, such as metal oxides.

Thus, in the case of aluminum, the particle can comprise alumina Al₂O₃ in a proportion, for example, of 10% per 90% of Al metal.

The metal of which the particles are predominantly composed may be chosen from, for example, transition metals, rare earth metals and their alloys and mixtures.

For example, the metal may be chosen from aluminum, copper, silver, gold, indium, iron, platinum, nickel, molybdenum, titanium, tungsten, antimony, palladium, zinc, tin and their alloys and mixtures.

In the preceding list, the noble metals and copper may, for example, be used. The term “noble metals” is understood to mean gold, silver, palladium, platinum and their alloys and mixtures.

In one embodiment, metal that may be used in accordance with the disclosure comprises silver.

For example, according to the disclosure, the loss in sheen, very probably due to the surface oxidation of a fine layer of silver deposited at the surface of the keratinous fiber, is avoided by virtue of the passivation, protection or stabilization provided, for example, by a fatty acid. This passivation, protection or stabilization is increased when use is made, as described later, of coated particles comprising a shell surrounding a core formed by the passivated particle.

According to the dislcosure, the carboxylate groups of formula (I) may be chosen, for example, from carboxylate groups derived from fatty acids, perfluoro(having 6 to 39 carbons, such as 10 to 16 carbons)alkylcarboxylates and hydroxy(having 6 to 39 carbons, such as 10 to 16 carbons)alkylcarboxylates. The carboxylate groups may, for example, be chosen from the myristate, stearate, oleate, behenate and tetradecanoate groups.

Generally, the R groups of the carboxylates are directed radially towards the outside of the particle.

The particles according to the disclosure are capable of being obtained by thermal decomposition of a carboxylate of the metal comprising the particle, the metal carboxylate corresponding to the formula (II): 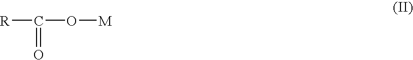
where R is as defined above and M is the metal constituting the particle.

This carboxylate is generally chosen from carboxylates derived from fatty acids, perfluoro(having from 6 to 39 carbons, such as from 10 to 16 carbons)alkylcarboxylates and hydroxy(having from 6 to 39 carbons, such as from 10 to 16 carbons)alkylcarboxylates.

The carboxylates may be chosen from, for example, the myristate, the stearate, the oleate, the behenate and the tetradecanoate.

In one embodiment, M comprises silver, and the carboxylate of formula (II) comprises a compound chosen from silver myristate, silver stearate, silver oleate, silver behenate, silver tetradecanoate and a silver perfluoro, having from 6 to 39 carbons, such as from 10 to 16 carbons, alkylcarboxylate.

The thermal decomposition is generally carried out at a temperature ranging from 100 to 800° C., such as from 150 to 500° C., in an inert atmosphere, for example a nitrogen atmosphere. The product obtained is washed, for example with an alkanol, and dispersed, for example in a nonpolar solvent, in order to remove the unreacted starting reactants. It is subsequently separated, for example by filtration, and finally dried. Nanoparticles are thus obtained where the metal is surrounded by the carboxylates with the alkyl chains projecting towards the outside of the particle and forming a monolayer with a thickness, for example, of 1 to 2 nm at the surface of the particles. This process makes it possible to obtain particles with a size as defined above and additionally exhibiting a low size polydispersity.

For further details with regard to such a process for the manufacture of metal nanoparticles, reference may be made to the following documents:
Two-dimensional Arrangement of Silver Nanoparticles on Water Surface, Koji A B E et al., Mol. Cryst. Liq. Cryst., 1998, Vol. 322, pp. 173-178;
Two-dimensional Array of Silver Nanoparticles, Koji A B E et al., Thin Solid Films, 327-329 (1998) 524-527;
Optical Properties of Dispersion and Monolayer of Silver Nanoparticles, Koji A B E et al. Mol. Cryst. Liq. Cryst., 1999, Vol. 327, pp. 34-36; and
Perfluorocarbon-stabilized Silver Nanoparticles Manufactured from Layered Silver Carboxylate, A. J. Lee et al., Chem Commun., 2002, 442-443.

In one embodiment, the particles incorporated in the cosmetic compositions disclosed herein are coated particles which comprise a core and a solid shell (core-shell structure), the core predominantly comprising at least one metal and being treated or passivated at its outer surface with carboxylate groups as described above. For example, the core may be surrounded, coated or encapsulated by a solid shell, wherein the size of the particles range from 1 nm to 500 nm.

Such passivated and coated particles have never been disclosed in the prior art and, surprisingly, further increase the effects of disclosed herein.

The shell can be comprised of an organic material or else of an inorganic material bonded to the core by a noncovalent bond.

According to a first embodiment of the disclosure, the shell surrounding the core is comprised of an organic material.

The term “organic material” is also understood to mean any material comprising at least one carbon atom.

According to the disclosure, this organic material is a material which is solid at ambient temperature and at atmospheric pressure.

The organic material can thus be chosen from organic polymers and oligomers, whether natural compounds or synthetic compounds.

The polymers or oligomers of the invention can be obtained by radical polymerization or by polycondensation.

The organic material can thus be chosen from poly(vinyl alcohol)s, poly(lactic acid)s, poly(glycolic acid)s, copolymers of lactic acid and of glycolic acid, polystyrenes, poly(methyl (meth)acrylate)s, acrylic and methacrylic copolymers, polyamides, polyesters, polyurethanes and polyureas.

The organic material can also be chosen from cellulose and derivatives of the latter, such as alkyl- and hydroxyalkylcelluloses for example methylcellulose, ethylcellulose and hydroxyalkylcellulose, or such as cellulose esters, for example cellulose acetate phthalate.

Finally, the organic material can be chosen from gelatin, pectin, optionally crosslinked, for example with glutaraldehyde, or a polysaccharide, such as carrageenan.

One organic material as disclosed herein is a styrene/methacrylic acid copolymer.

According to a second embodiment of the invention, the shell surrounding the core is comprised of an inorganic material chosen from metal oxides and organometallic polymers.

The shell or the capsule comprised of an organic or inorganic material generally has a thickness ranging from 2 to 300 nm, for example 5 to 250 nm, such as 10 to 100 nm.

It should be noted that this shell or this capsule, and in accordance with the well-known definition of encapsulation in the technical field, is not a monolayer or a molecular layer but actually a layer which may be described as a “thick” wall, the thickness of which is generally within the range defined above.

According to the disclosure, the capsule, coating or shell of organic or inorganic material is generally connected to the core by a physical bond, without the need for covalent bonds. In other words, in the particles incorporated in the compositions disclosed herein, the core/shell interface does not require covalent bonds.

The shell or capsule of organic material around the metal core (composed of the metal particle passivated by the carboxylates) in the coated particles of the compositions disclosed herein can be formed by various processes.

These processes, which are generally denoted by the terms of encapsulation or nanoencapsulation process, are known to a person skilled in the art in this technical field and can be generally divided into two main families: physicochemical processes and chemical processes.

The physicochemical processes can be chosen from phase separation or coacervation, evaporation, solvent extraction, thermal gelling, controlled precipitation and any other known physicochemical process for microencapsulation.

The chemical processes can be chosen from interfacial polycondensation, interfacial polymerization, polymerization in dispersed medium, in situ polycondensation, emulsion polymerization and any other known chemical process for microencapsulation.

The physicochemical and chemical processes are described in detail below but reference may also be made to the document Microencapsulation Methods and Industrial Applications, (ISBN 0-8247-9703-S).

Mention may be made, among the various encapsulation processes which can be used, of encapsulation by emulsion polymerisation, such as encapsulation by emulsion polymerization as described in the paper Preparation of Polymer Coated Functionalised Silver Nanoparticles (J. Am. Chem. Soc., 1999, 121, 10642-10643).

As is described in the preceding paper, the encapsulation of the nanoparticles is obtained by a conventional radical emulsion polymerization process. However, in order to obtain specific polymerization on the surface of the metal nanoparticles, the micelle phase of the emulsion comprises an amphiphilic compound or a mixture of amphiphilic compounds, the polar part or the hydrophobic part of which has affinity to the metal surface.

Such a process makes it possible to obtain a core-shell system comprised of a metal core (comprised of a metal particle passivated by carboxylates) and a polymer capsule with a thickness of generally greater than 2 nm such as from 2 nm and 300 nm.

Mention may be made, among the various constituents which can be made use of in carrying out the emulsion polymerization described above, to:
Water and/or ethanol or their mixtures as continuous phase of the emulsion. In one embodiment, the continuous phase comprises water.
Amphiphilic molecules, the polar part of which is comprised of one or more carboxyl functional groups. Among amphiphilic molecules, mention may be made of fatty acids such as oleic acid.
A mixture of styrene and of methacrylic acid as precursor monomers of the capsule.
Passivated silver nanoparticles as support to be encapsulated.

The outer surface of the passivated and coated particles, that is to say the outer surface of the capsule or of the shell, can be covalently modified by at least one chemical group which is capable of improving the adsorption of the particles on keratinous substances, such as the hair. This surface can also be covalently modified by at least one chemical group capable of reacting chemically with keratinous substances, such as the hair.

In the first case, the adsorption on keratinous substances, such as the hair, of the core-shell nanoparticles of the compositions disclosed herein can be improved by covalently modifying the capsule of organic or inorganic material, such as a polymer, with various chemical groups (Group A below) which may render the surface of the particles, for example, more hydrophobic and/or more cationic and/or more anionic and/or more hydrophilic.

The adsorption is defined as employing lower bonding energies than covalent bonds, that is to say less than 50 kcal/mol, between the keratinous substance, such as the individual hair, and the particle. These low-energy bonds may be, for example, Van der Waals forces, hydrogen bonds, electron donor-acceptor complexes, and the like.

The group capable of improving the adsorption of the particles on keratinous substances is generally chosen the following Group A:

Group A:
Carboxylic acids and their salts,
Primary, secondary, tertiary and quaternary amines,
Phosphates,
Sulphur oxides, such as sulphones, sulphonic, sulphoxides and sulphates, and
Aromatic rings, such as phenyl, triazine, thiophene and imidazole.

In the second case, the adhesion to keratinous substances, such as the hair, of the nanoparticles of the invention is obtained by covalently modifying the capsule of organic or inorganic material with various groups (Group B) capable of reacting chemically with the keratinous substance. The term “groups having a reactivity with regard to the keratinous substance, such as the individual hair,” is understood to mean the groups capable of forming a covalent bond with this substance, for example with the amines and/or the carboxylic acids and/or the thiols of the amino acids constituting the keratinous substance. The formation of these covalent bonds can either be spontaneous or can be carried out by activation such as by temperature, pH, light, a coreactant or a chemical or biochemical catalyst, such as an enzyme.

The group capable of reacting chemically or able to react chemically with keratinous substances, such as the hair, is generally chosen from the following Group B: Group B:
Epoxides,
Vinyl and activated vinyl: acrylonitrile, acrylic and methacrylic esters, crotonic acid and esters, cinnamic acid and esters, styrene and derivatives, butadiene, vinyl ethers, vinyl ketones, maleic esters, maleimides, vinyl sulphones, and the like,
Carboxylic acids and their derivatives: anhydride, acid chloride, esters,
Acetals, hemiacetals,
Aminals, hemiaminals,
Ketones and α-hydroxyketones, α-haloketones,
Lactones, thiolactones,
Isocyanates,
Thiocyanates,
Imines,
Imides (succinimides, glutimides),
Pyridyldithio,
N-Hydroxysuccinimide esters,
Imidates,
Oxazine and oxazoline,
Oxazinium and oxazolinium,
Groups of formula R₁X wherein
R₁ is chosen from an alkyl group having from 1 to 30 carbons, an aryl group having from 6 to 30 carbons, and an aralkyl group having from 7 to 30 carbons, the alkyl group having from 1 to 30 carbons; and
X is chosen from a leaving group such as 1, Br, Cl, and OSO₃R, where R is chosen from H and an alkyl group having from 1 to 30 carbons, —SO₂R′, where R′ is chosen from H and an alkyl group having from 1 to 30 carbons, a tosyl group, N(R″)₃, where R″ is chosen from an alkyl group having from 1 to 30 carbons, and OPO₃R′″₂, where R′″ is chosen from H and an alkyl group having from 1 to 30 carbons;
among the groups of formula R₁X, mention may be made of alkyl, aryl and aralkyl halides;
Groups of formula R₂X wherein
R₂ is chosen from carbon rings having from 3 to 30 carbons and unsaturated heterocycle with 3 to 20 ring members comprising one or more heteroatoms chosen from N, S, O and P, and
X comprises a leaving group as defined above.
Among the groups of formula R₂X, mention may be made of the halides of unsaturated rings, such as chlorotriazine, chloropyrimidine, chloroquinoxaline and chlorobenzotriazole,
Groups of formula R₃SO₂X, wherein
   R₃ has the same meaning as R₁ and
   X comprises a leaving group and has the meaning already given above,
Lactones,
Thiolactones, and
Siloxanes.

Non-limiting mention may be made, by way of example, of the activation by N-hydroxysulphosuccinimide of core-shell particles of silver/styrene-methacrylic copolymer type. The sulphosuccinimide functional groups are, in the case of an organic shell or capsule, grafted to the surface of the particles via the carboxyl groups which the polymer of the capsule possesses. Such a surface group makes it possible to covalently bond the nanoparticles described herein to the hair by reaction with the free surface amines which the hair fiber possesses. The reaction scheme below illustrates activation of the encapsulated passivated nanoparticles. 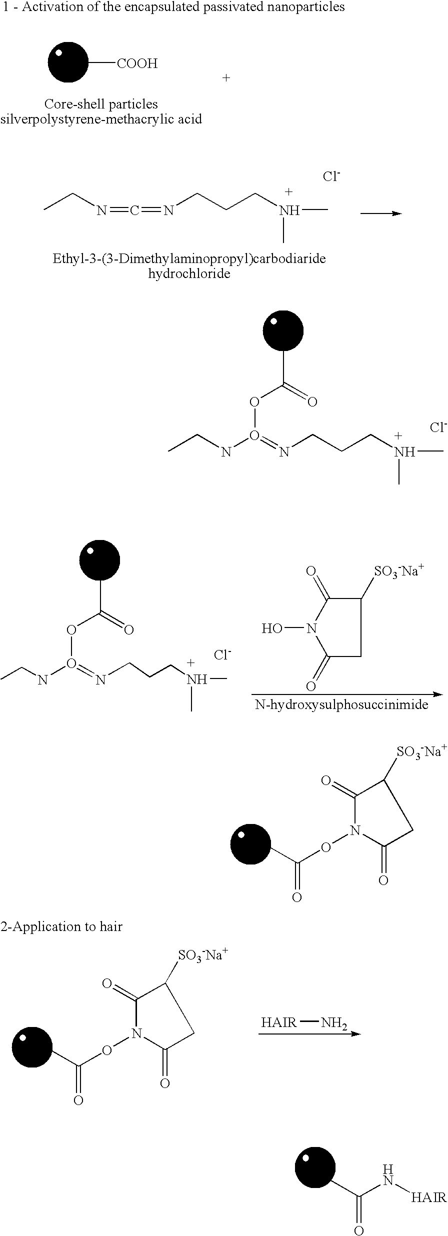
It should be noted that the chemical functional groups on the surface of the keratinous substance, for example of the hair fiber, can be increased in density by pretreatment of the fiber with a solution of polymer having a particular affinity for the fiber and exhibiting reactive functional groups. In the preceding example, the density of the amine functional groups at the surface of the fiber can be increased, for example, by absorbing polyethyleneimine beforehand.

In order to increase the durability of the effect over time, in addition to the improvement in the adhesion or in the absorption, it is possible to use metal particles (which, according to the disclosure, are passivated by carboxylate groups) encapsulated by a shell of reactive organic/inorganic or organic polymer capable of creating interparticle covalent bonds after evaporation of the solvent phase.

In the cosmetic compositions, the treated (passivated) and optionally encapsulated or coated metal nanoparticles of the disclosure are generally present in a concentration ranging from 0.0001% to 50%, for example from 0.01% to 5% such as from 0.05% to 2% by weight of the total weight of the composition.

The composition disclosed herein additionally comprises a physiologically acceptable medium. This term is used to denote a medium capable of being applied to keratinous fibers such as the hair of human beings.

The physiologically acceptable medium of the composition generally comprises at least one solvent. The solvent makes it possible, for example, to convey the passivated and optionally encapsulated metal nanoparticles. The solvent can be chosen generally from organic solvents, water and their mixtures.

The organic solvents are generally chosen from C₁ to C₄ aliphatic alcohols, such as ethanol and isopropanol, polyols, such as glycerol or propylene glycol, aromatic alcohols, such as benzyl alcohol, alkanes, such as C₅ to C₁₀ alkanes, acetone, methyl ethyl ketone, methyl acetate, butyl acetate, alkyl acetate, dimethoxyethane, diethoxyethane and their mixtures.

The compositions disclosed herein can be packaged in various forms such as in an aerosol device.

The composition disclosed herein can further comprise a propellant. The propellant comprises compressed or liquefied gases commonly employed for the preparation of aerosol compositions. Use will may be made of air, carbon dioxide gas, compressed nitrogen or a soluble gas, such as dimethyl ether, halogenated (such as fluorinated) or nonhalogenated hydrocarbons, and their mixtures.

The compositions disclosed herein can additionally comprise conventional cosmetic additives chosen from a nonexhaustive list, such as reducing agents, oxidizing agents, thickening agents, softeners, antifoaming agents, direct and oxidation dyes, pearlescent agents, fragrances, peptizing agents, preservatives, and anionic and amphoteric surfactants.

The cosmetic composition disclosed herein can be a cosmetic treatment composition, such as a composition for contributing sheen to keratinous substances. In one embodiment, it is a hair cosmetic composition.

The hair cosmetic compositions disclosed herein, after application to the hair, can be rinsed out or left in. The compositions, such as the hair compositions, can be provided in various dosage forms (formulations), such as a lotion, spray, foam, lacquer, conditioner or a shampoo, for example.

The invention will be better understood with the help of the nonlimiting illustrative examples which follow and which constitute non-limiting embodiments of the composition disclosed herein. In the examples, the percentages are expressed by weight and a.m. means active material.

### COMPARATIVE EXAMPLE

In this example, a composition according to the present disclosure and a composition according to the prior art were prepared.

Composition 1 (according to the invention):

| | |
|---|---|
| Passivated silver nanoparticles⁽¹⁾ | 0.1% (a.m.) |
| Cyclopentasiloxane⁽²⁾ | 10% |
| Ethanol | 5% |
| C₁₂-C₁₅ Alkyl benzoate⁽³⁾ | 1% |
| Fragrance | q.s. |
| Isododecane⁽⁴⁾ | q.s. for 100% |

⁽¹⁾The silver nanoparticles were obtained^{[a,b]} by thermal decomposition (250° C.) under nitrogen of silver myristate^{[c]}. The residue obtained was washed with ethanol, filtered off, dried and then dissolved at a concentration of 1% in isododecane^{[4]}. As was shown by the transmission electron microscopy (TEM) exposures taken, the silver nanoparticles synthesized exhibited a diameter ranging from 5 nm to 20 nm.
⁽²⁾Sold by Dow Corning under the trade name Dow Corning 1501 Fluid
⁽³⁾Sold by Witco under the trade name Finsolv TN
⁽⁴⁾Sold by Permethyl under the trade name Permethyl 99A
^{[a]}Two-dimensional Arrangement of Silver Nanoparticles on Water Surface, Koji ABE et al., Mol. Cryst. Liq. Cryst., 1998, Vol. 322, pp. 173-178.
^{[b]}Optical Properties of Dispersion and Monolayer of Silver Nanoparticles, Koji ABE et al., Mol. Cryst. Liq. Cryst., 1999, Vol. 327, pp. 34-36.
^{[c]}Two-dimensional Array of Silver Nanoparticles, Koji ABE et al., Thin Solid Films, 327-329 (1998) 524-527.

Composition 2 (according to the prior art):

| | |
|---|---|
| Silver nanoparticles⁽⁵⁾ | 0.1% (a.m.) |
| Cyclopentasiloxane⁽²⁾ | 10% |
| Ethanol | 5% |
| C₁₂-C₁₅ Alkyl benzoate⁽³⁾ | 1% |
| Fragrance | q.s. |
| Isododecane⁽⁴⁾ | q.s. for 100% |

⁽⁵⁾Silver nanoparticles sold under the reference “Nanocrystalline Silver Dispersion” by Nanophase Technologies. As was shown by the transmission electron microscopy (TEM) exposures taken, the nanoparticles exhibited a diameter ranging from 5 nm to 20 nm.

Each of the preceding compositions was applied to a lock of brown hair weighing 2.7 g (European hair with a length of 20 cm) at the rate of one gram of composition per lock. After application, the locks were dried under a hairdryer (70° C.) for 30 minutes.

A measurement of sheen was subsequently carried out on a batch of 10 locks treated as indicated above with one or other of the compositions.

The sheen was determined using a photogoniometer by measuring the specular and diffuse reflections of the locks of hair laid flat on a support. Using a 175 watt xenon arc lamp (model ORC175F) coupled to a V filter (lambda), light was emitted over the lock under an angle of +30° C. with respect to the normal to its surface. Using a movable receiving arm, the specular reflection (R), corresponding to the maximum light intensity reflected in the vicinity of an angle of −30°, and the diffuse reflection (D), corresponding to the light reflected at an angle of +15° C., were measured. According to the disclosure, the sheen was determined by calculating the ratio (R)/(D).

In order to evaluate the retention of the sheen over time, the measurement of sheen was repeated on the same treated locks after storing the latter for one month under ambient conditions (20° C. and 50% RH).

The results obtained in terms of stability of the sheen are given in the following Table III:

TABLE III

| | Sheen | | |
|---|---|---|---|
| | before | Sheen immediately | Sheen of the treated |
| | treatment | after treatment | locks after 1 month |
| Composition 1 | 25 ± 2 | 34 ± 2 | 31 ± 4 |
| (invention) | | | |
| Composition 2 | 24 ± 3 | 30 ± 3 | 25 ± 3 |
| (prior art) | | | |

As shown in the preceding Table III, it was recorded that the composition disclosed herein retains these sheen properties after storage of the locks under ambient conditions for 1 month. By way of comparison, the composition of the prior art loses almost all its reflectivity after storage of the locks under ambient conditions for 1 month.

1. A cosmetic composition comprising, in a physiologically acceptable medium, passivated particles predominantly comprising at least one metal, wherein the particles are treated at their outer surface with at least one carboxylate group of formula (I): 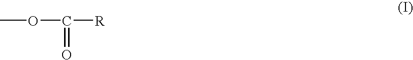
wherein R comprises a linear hydrocarbon having from 6 to 40 carbons, and the particles are less than or equal to 500 nm in size.

2. The composition according to claim 1, wherein R comprises at least one ethylenic double bond.

3. The composition according to claim 1, wherein R is substituted by at least one substituent chosen from hydroxyls and halogens.

4. The composition according to claim 1, wherein R comprises a linear hydrocarbon substituted by at least one fluorine atom.

5. The composition according to claim 1, further comprising an agent exhibiting a cosmetic activity chosen from:
saccharides, oligosaccharides and polysaccharides which may or may not be hydrolysed and which may or may not be modified,
amino acids, oligopeptides, peptides, proteins, which may or may not be hydrolysed and which may or may not be modified, poly(amino acids) and enzymes,
branched and unbranched fatty acids and alcohols,
animal, vegetable and mineral waxes,
ceramides and pseudoceramides,
hydroxylated organic acids,
UV screening agents,
antioxidants and agents for combating free radicals,
chelating agents,
antidandruff agents,
seborrhoea-regulating agents,
soothing agents,
cationic surfactants,
cationic and amphoteric polymers,
organomodified and nonorganomodified silicones,
mineral, vegetable and animal oils,
polyisobutenes and poly(α-olefin)s,
esters,
soluble and dispersed anionic polymers,
soluble and dispersed non-ionic polymers,
reducing agents,
coloring agents and coloring materials,
foaming agents,
film-forming agents,
particles which are not passivated or treated,
and their mixtures.

6. The composition according to claim 5, wherein the coloring agents and coloring materials comprise hair dyes.

7. The composition according to claim 5, wherein the agent exhibiting a cosmetic activity is present in a proportion ranging from 0.001 to 10% by weight of the cosmetic composition.

8. The composition according to claim 7, wherein the agent exhibiting a cosmetic activity is present in a proportion ranging from 0.01 to 5% by weight of the cosmetic composition.

9. The composition according to claim 0.1, wherein the particle size ranges from 1 nm to 500 nm.

10. The composition according to claim 9, wherein the particle size ranges from 1 nm to 100 nm.

11. The composition according to claim 10, wherein the particle size ranges from 1 nm to 50 nm.

12. The composition according to claim 1, wherein the particles have a shape chosen from spheres, flakes, fibers, tubes, and polyhedra.

13. The composition according to claim 1, wherein the particles comprise at least 80% by weight of the at least one metal.

14. The composition according to claim 13, wherein the particles comprise at least 90% by weight of at the least one metal.

15. The composition according to claim 13, wherein the particle comprises 100% by weight of the at least one metal.

16. The cosmetic composition according to claim 1, wherein the at least one metal is chosen from aluminum and all the metals with an atomic number ranging from 21 to 82, and the alloys thereof.

17. The composition according to claim 16, wherein the particles comprise a mixture of two or more of the metals and/or metal alloys.

18. The composition according to claim 1, wherein the particles are chosen from composite particles comprising more than one region with adjacent regions comprising different metals, alloys or mixtures thereof.

19. The composition according to claim 18, wherein the particles comprise multilayer composite particles comprising an inner core comprising
at least one metal, alloy or mixture thereof, at least partially covered by a first layer comprising a metal, metal alloy or mixture thereof which is different from that constituting the inner core and optionally by one or more other layers, each of these layers at least partially covering the preceding layer and each of these layers comprising a metal, alloy or mixture thereof, which is different from the following layer and from the preceding layer.

20. The composition according to claim 11, wherein the particles further comprise stabilizers and unavoidable impurities.

21. The composition according to claim 1., wherein the particles further comprise metal compounds.

22. The composition according to claim 11 wherein the at least one metal is chosen from transition metals, rare earth metals, alloys and mixtures thereof.

23. The composition according to claim 1., wherein the at least one metal is chosen from aluminum, copper, silver, gold, indium, iron, platinum, nickel, molybdenum, titanium, tungsten, antimony, palladium, zinc, tin, alloys and mixtures thereof.

24. The composition according to claim 23, wherein the at least one metal is chosen from gold, silver, palladium, platinum, alloys and mixtures therof

25. The composition according to claim 24, wherein the at least one metal is silver.

26. The composition according to claim 1, wherein the carboxylate groups are chosen from carboxylate groups derived from fatty acids, perfluoroalkylcarboxylates having from 6 to 39 carbons, and hydroxyalkylcarboxylates having from 6 to 39 carbons.

27. The composition according to claim 26, wherein the perfluoroalkylcarboxylates have from 10 to 16 carbons.

28. The composition according to claim 26, wherein the hydroxyalkylcarboxylates have from 10 to 16 carbons.

29. The composition according to claim 26, wherein the carboxylate groups are chosen from myristate, stearate, oleate, behenate and tetradecanoate groups.

30. The composition according to claim 1, wherein the R groups of the at least one carboxylate are directed radially towards the outside of the particle.

31. The composition according to claim 1; wherein the particles are capable of being obtained by thermal decomposition of a carboxylate of the at least one metal, wherein the metal carboxylate corresponds to the formula (II): 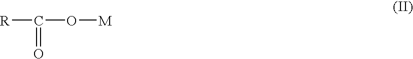
wherein R comprises a linear hydrocarbon having from 6 to 40 carbons, and M comprises at least one metal.

32. The composition according to claim 31, wherein the carboxylate is chosen from carboxylates derived from fatty acids, perfluoroalkylcarboxylates having from 6 to 39 carbons and hydroxyalkylcarboxylates having from 6 to 39 carbons.

33. The composition according to claim 32, wherein the perfluroalkylcarboxylates have from 10 to 16 carbons.

34. The composition according to claim 32, wherein the hydroxyalkylcarboxylates have from 10 to 16 carbons.

35. The composition according to claim 32, wherein the carboxylate is chosen from myristate, stearate, oleate, behenate and tetradecanoate.

36. The composition according to claims 32, wherein the metal M is silver and the carboxylate of formula (II) is chosen from silver myristate, silver stearate, silver oleate, silver behenate, silver tetradecanoate, and a silver perfluoroalkylcarboxylate having from 6 to 39 carbons.

37. The composition according to claim 36, wherein the perfluoroalkylcarboxylate has from 10 to 16 carbons.

38. The composition according to claim 31., wherein the thermal decomposition is carried out at a temperature ranging from 100 to 800° C. in an inert atmosphere.

39. The composition according to claim 38, wherein the thermal decomposition is carried out at a temperature ranging from 100 to 500° C.

40. The composition according to claim 1, wherein the particles are coated particles comprising a core and a solid shell, the core being passivated particles predominantly comprising at least one metal, wherein the particles are treated at their outer surface with at least one carboxylate group of formula (I): 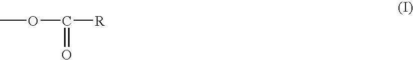
wherein R comprises a linear hydrocarbon having from 6 to 40 carbons, and the particles are less than or equal to 500 nm in size.

41. The composition according to claim 40, wherein the shell comprises at least one organic or at least one inorganic material bonded to the core by a noncovalent bond.

42. The composition according to claim 41, wherein the at least one organic material is chosen from organic polymers and oligomers.

43. The composition according to claim 42, wherein the organic material is chosen from poly(vinyl alcohol)s, poly(lactic acid)s, poly(glycolic acid)s, copolymers of lactic acid and of glycolic acid, polystyrenes, poly(methyl(meth)acrylate)s, acrylic and methacrylic copolymers, polyamides, polyesters, polyurethanes and polyureas.

44. The composition according to claim 42, wherein the at least one organic material is chosen from cellulose and its derivatives.

45. The composition according to claim 44, wherein the at least one organic material is chosen from alkyl- and hydroxyalkylcelluloses.

46. The composition according to claim 45, wherein the at least one organic material is chosen from methylcellulose, ethylcellulose, and hydroxyalkylcellulose.

47. The composition according to claim 44, wherein the at least one organic material is chosen from cellulose esters.

48. The composition according to claim 47, wherein the at least one organic material comprises cellulose acetate phthalate.

49. The composition according to claim 41, wherein the at least one organic material is chosen from gelatin and pectin, wherein said pectin is optionally crosslinked.

50. The composition according to claim 49, wherein the at least organic material is a pectin crosslinked with a glutaraledhyde or a polysaccharide.

51. The composition according to claim 50, wherein the polysaccharide comprises carrageenan.

52. The composition according to claim 43, wherein the at least one organic material comprises a styrene-methacrylic acid copolymer.

53. The composition according to claim 41, wherein the at least one inorganic material is chosen from metal oxides and organometallic polymers.

54. The composition according to claim 41, wherein the shell of the particles has a thickness ranging from 2 to 300 nm.

55. The composition according to claim 54, wherein the shell of the particles has a thickness ranging from 5 to 250 nm.

56. The composition according to claim 55, wherein the shell of the particles has a thickness ranging from 10 to 100 nm.

57. The composition according to claim 41, wherein the outer surface of the particles is covalently modified by at least one chemical group which is capable of improving the adsorption of the particles on keratinous substances.

58. The composition according to claim 41, wherein the outer surface of the particles is covalently modified by at least one chemical group capable of reacting chemically with keratinous substances.

59. The composition according to claim 57, wherein the at least one chemical group capable of improving the adsorption of the particles on keratinous substances is chosen from the following groups:
Carboxylic acids and their salts,
Primary, secondary, tertiary and quaternary amines,
Phosphates,
Sulphur oxides, sulphonic acids, sulphoxides and sulphates,
Aromatic rings, triazine, thiophene and imidazole.

60. The composition according to claim 59, wherein the at least one chemical group capable of improving the adsorption of the particles on keratinous substances comprises a sulphone.

61. The composition according to claim 59, wherein the at least one chemical group capable of improving the adsorption of the particles on keratinous substances comprises a phenyl.

62. The composition according to claim 58, wherein the at least one chemical group capable of reacting chemically with keratinous substances is chosen from the following groups:
Epoxides,
Vinyl and activated vinyl: acrylonitrile, acrylic and methacrylic esters, crotonic acid and esters, cinnamic acid and esters, styrene and derivatives, butadiene, vinyl ethers, vinyl ketones, maleic esters, maleimides, vinyl sulphones, and the like,
Carboxylic acids and their derivatives,
Acetals, hemiacetals,
Aminals, hemiaminals,
Ketones and α-hydroxyketones, α-haloketones,
Lactones, thiolactones,
Isocyanates,
Thiocyanates,
Imines,
Imides,
Pyridyldithio,
N-Hydroxysuccinimide esters,
Imidates,
Oxazine and oxazoline,
Oxazinium and oxazolinium,
Groups of formula R₁X wherein
R₁ is chosen from alkyl groups having 1 to 30 carbons, aryl groups having 6 to 30 carbons, and aralkyl groups having 7 to 30 carbons, and
X is a leaving in group,
Groups of formula R₂X wherein
R₂ is chosen from carbon rings having 3 to 30 carbons and an unsaturated heterocycle with 3 to 20 ring members comprising one or more heteroatoms chosen from N, S, O and P, and
X is a leaving group,
Groups of formula R₃SO₂X, wherein
R₃ is chosen from alkyl groups having 1 to 30 carbons, aryl groups having 6 to 30 carbons, and aralkyl groups having 7 to 30 carbons and X represents a leaving group,
Lactones,
Thiolactones, and
Siloxanes.

63. The composition according to claim 62, wherein the vinyl and activated vinyl groups are chosen from acrylonitrile, acrylic and methacrylic esters, crotonic acid and esters, cinnamic acid and esters, styrene and derivatives, butadiene, vinyl ethers, vinyl ketones, maleic esters, maleimides, and vinyl sulphones.

64. The composition according to claim 62, wherein the carboxylic acids and derivatives thereof are chosen from anhydrides, acid chlorides, and esters.

65. The composition according to claim 62, wherein the imides are chosen from succinimides and glutimides.

66. The composition according to claim 60, wherein the leaving group (X) in the groups of the formulae R₁X, R₂X, and R₃SO₂X is chosen from I, Br, Cl, OSO₃R, wherein R is chosen from H and alkyl groups having from 1 to 30 carbons, —SO₂R′, wherein R′ is chosen from H and alkyl groups having from 1 to 30 carbons, a tosyl group, N(R″)₃, wherein R″ is chosen from alkyl groups having from 1 to 30 carbons, and OPO₃R′″₂, wherein R′″ is chosen from H and alkyl groups having from 1 to 30 carbons.

67. The composition according to claim 66, wherein X in R₂X, is a leaving group chosen from chlorotriazine, chloropyrimidine, chloroquinoxaline and chlorobenzotriazole,

68. The composition according to claim 1, wherein the particles are present in the composition at a concentration ranging from 0.0001% to 50% by weight relative to the total weight of the composition.

69. The composition according to claim 68, wherein the particles are present in the composition at a concentration ranging from 0.01% to 5% by weight relative to the total weight of the composition.

70. The composition according to claim 69, wherein the particles are present in the composition at a concentration ranging from 0.05% and 2% by weight relative to the total weight of the composition.

71. The composition according to claim 1, wherein the physiologically acceptable medium comprises at least one solvent.

72. The composition according to claim 71, wherein the at least one solvent is chosen from organic solvents, water and their mixtures.

73. The composition according to claim 72, wherein the organic solvents are chosen from C₁ to C₄ aliphatic alcohols, polyols, aromatic alcohols, alkanes, acetone, methyl ethyl ketone, methyl acetate, butyl acetate, alkyl acetate, dimethoxyethane, diethoxyethane and their mixtures.

74. The composition according to claim 73, wherein the C₁ to C₄ aliphatic alcohols are chosen from ethanol and isopropanol.

75. The composition according to claim 73, wherein the polyols are chosen from glycerol and propylene glycol.

76. The composition according to claim 73, wherein the aromatic alcohols comprise benzyl alcohol.

77. The composition according to claim 713, wherein the alkanes are chosen from C₅ to C₁₀ alkanes.

78. The composition according to claim ˜1, the composition further comprising cosmetic additives chosen from reducing agents, oxidizing agents, thickening agents, softeners, antifoaming agents, direct dyes and oxidation dyes, pearlescent agents, fragrances, peptizing agents, preservatives, and anionic and amphoteric surfactants.

79. A cosmetic treatment composition comprising, in a physiologically acceptable medium, passivated particles predominantly comprising at least one metal, wherein the particles are treated at their outer surface with at least one carboxylate group of formula (I): 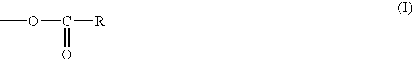
wherein R comprises a linear hydrocarbon having from 6 to 40 carbons, and the particles are less than or equal to 500 nm in size.

80. The cosmetic treatment composition according to claim 79, wherein the cosmetic treatment composition contributes sheen to keratinous substances.

81. The cosmetic treatment composition according to claim 79, which is a hair composition that contributes sheen to the hair.

82. The composition according to claim 81, wherein the hair composition is in the form chosen from a lotion, a spray, a foam, a lacquer, a conditioner, and a shampoo.

83. A composition comprising, in a physiologically acceptable medium, passivated particles predominantly comprising at least one metal, wherein the particles are treated at their outer surface with at least one carboxylate group of formula (I): 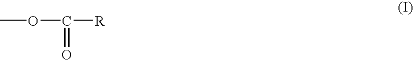
wherein R comprises a linear hydrocarbon having from 6 to 40 carbons, and the particles are less than or equal to 500 nm in size, which is packaged in an aerosol device.

84. Coated particles comprising a core and a solid shell, the core being passivated particles predominantly comprising at least one metal, wherein the particles are treated at their outer surface with at least one carboxylate group of formula (I): 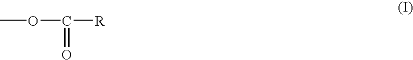
wherein R comprises a linear hydrocarbon having from 6 to 40 carbons, and the particles are less than or equal to 500 nm in size.

85. A cosmetic process for the treatment of keratinous substances comprising the application to keratinous substances, a composition comprising, in a physiologically acceptable medium, passivated particles predominantly comprising at least one metal, wherein the particles are treated at their outer surface with at least one carboxylate group of formula (I): 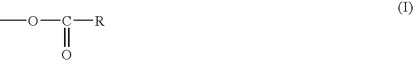
wherein R comprises a linear hydrocarbon having from 6 to 40 carbons, and the particles are less than or equal to 500 nm in size,

86. The cosmetic process according to claim 85, wherein the keratinous substances are hair and the process contributes sheen to the hair.

87. The composition according to claim 21, wherein the particles further comprise metals oxides.
